(19) United States
(12) Patent Application Publication
(10) Pub. No.: US 2013/0231533 A1
(43) Pub. Date: Sep. 5, 2013

(54) MEDICAL APPLICATIONS OF A MINIATURE VIDEOSCOPE

(71) Applicants: Stephanos Papademetriou, Sunnyvale, CA (US); Theofilos Kotseroglou, Hillsborough, CA (US)

(72) Inventors: Stephanos Papademetriou, Sunnyvale, CA (US); Theofilos Kotseroglou, Hillsborough, CA (US)

(21) Appl. No.: 13/479,202

(22) Filed: May 23, 2012

Related U.S. Application Data
(60) Provisional application No. 61/519,415, filed on May 23, 2011.

(51) Int. Cl. A61B 1/00; A61B 1/05; A61B 1/06

(52) U.S. Cl. CPC A61B 1/00043; A61B 1/06; A61B 1/05; A61B 1/00103
USPC 600/110; 600/109; 600/101

(57) ABSTRACT

Endoscopes, particularly videoscopes, of small size and profile are applied to various medical devices such as intravascular catheters and feeding tubes. The endoscopes are of high flexibility, good resolution and low cost so as to be disposable after use.

This application claims benefit of provisional application Ser. No. 61/519,415, filed May 23, 2011.

### BACKGROUND OF THE INVENTION

This invention relates generally to the construction of low cost (both patient contact and hardware), miniature in size, high flexibility, good resolution videoscopes that include illumination and access ports.

Because endoscopic imaging requires direct visualization of internal organ surfaces, both illumination and detection elements must be ported through an often tortuous anatomical geometry to a region of interest. As such, the accessibility of internal organs is dictated by both the size and rigidity of the endoscope.

Conventional flexible endoscopes are approximately the same thickness as a human finger and are primarily designed for high-quality color imaging. Relatively wide field of view imaging (>70° FOV) is necessary for navigating the scope within the body, inspecting tissue, diagnosing disease, and guiding surgical interventions.

Flexible endoscopy was ushered in by Optical Coherent Fiberoptic Bundles (OCFB), which serve as bendable conduits for transmitting light between proximal and distal ends of the endoscope (B. I. Hirschowitz, L. E. Curtiss, C. W. Peters and H. M. Pollard, “Demonstration of a new gastroscope, the “fiberscope”,” Gastroenterology 35, pp. 50-53, (1958)). OCFB technology is still in use today, but more modern incarnations often employ a proximal video camera for image capture and subsequent display on a video monitor. By matching endoscope mechanical properties to those of the target organ, the risk of perforation by submillimeter diameter scopes can be avoided. Leached OCFB technologies contain a nonfused length between distal and proximal ends, and were developed to alleviate durability and flexibility issues. These leached fiber bundles (us.schott.com/lightingimaging/english/life-science/medical-products/transmitting-images.html) do exhibit some superior mechanical properties, but 2.5 to 3 times lower core density than nonleached OCFB's and higher cost deter their acceptance.

These optical and mechanical limitations of OCFB devices explain, in large part, why ultrathin flexible endoscopes are only minimally sufficient and not routinely used for medical procedures. Albeit there is great potential for performing less-invasive procedures in previously inaccessible regions of the human body if limitations in image quality and endoscope flexibility and overall size can be overcome.

Most flexible endoscopes are comprised of miniature Charge-Coupled Device (CCD) or Complementary Metal-Oxide-Semiconductor (CMOS) video chips that have been placed at the distal tip of the flexible shaft, using incoherent optical fiber bundles to deliver white-light diffuse illumination (J. Baillie, “The Endoscope,” Gastrointest. Endosc. 65, 886-893 (2007)). To accommodate imaging within small vessels, lumens, and ducts within the human body, ultrathin endoscopes have been developed by reducing the overall device diameter and reducing the pixel size of the digital sensor (so that image resolution does not suffer too much as the size of the sensor gets smaller). Having said that, there are many application in medicine where high resolution is not the important factor, rather low cost, small size (<2 mm or even <1 mm), and high flexibility (<5 mm bend radius) are the desired device attributes.

### IN SUMMARY

Standard OCFB, are too stiff and rigid when they offer higher image resolutions. At smaller sizes of less than 1 mm OD, they start becoming more flexible, but they suffer highly reduced resolution (<10,000 pixels). Even at these smaller sizes they can be too stiff to reach smaller anatomical vessels, and ducts in the human body.

Leachable OCFBs offer superior mechanical properties, but at a highly reduced resolution and much higher overall cost. They typically result in more expensive, highly flexible, larger devices with reduced resolution compared to their nonleachable counterparts.

Scanning approaches to endoscopy imaging offer smaller constructs (typically more than 2 mm in OD but also as small as 1.2 mm in OD) with higher resolutions than those offered by OCFBs, but at much higher cost for both the endoscope (patient contact portion of the device) as well as the opto-electronics, hardware and software on the capital equipment side for image processing and illumination. Although these technologies may offer viable solutions for applications such as high-resolution optical biopsies, cellular imaging, and in-vivo fluorescence microscopy to name a few, they can become highly prohibitive in many other practical applications, where cost, size, and flexibility are far more important than high resolution. Due to their complexity (and resultant high cost), they could never become truly single-use medical device imaging solutions.

Finally, videoscopes have the potential to bridge the gap of low cost, flexibility, small size, as an acceptable resolution. For example Medigus Ltd., Omer, Israel, offers video cameras (medigus.com/MicroCamerasOverview/Cameras.aspx) from 1.2 mm OD to 3.0 mm OD.

The resolution of the digital camera is a function of the size of the pixel. Thus smaller size chips can be made without suffering from reduced resolution (due to their smaller size) if their pixel can be made smaller. Smaller video digital CMOS chips with small enough pixels can be made that could easily be less than even 1 mm (for example several products offered by Omnivision Inc. nowadays are made with pixel sizes as small as 1.1 μm, and soon to be made with pixels as small as 0.9 μm).

### PRINCIPALS OF THE INVENTION

Digital cameras (using CCD or CMOS technology) have been miniaturized enough to be utilized as videoscopes at the distal end of an endoscope. Illumination for such videoscopes is provided by an LED or a multitude of LED's attached at the distal end of the endoscope or by fibers that terminate at the distal end of the structure along with the aforementioned digital imaging sensor.

All of the imaging technology options discussed so far can offer a viable miniature scope for the purposes of this patent application. The desirable properties of imaging scope of interest are attainable with current technologies, can be constructed by someone knowledgeable in the art, and are summarized below:

The imaging scope of interest must simultaneously offer the following attributes: (1) a miniature size (depending on the device construct disclosed in this application, from <1.7 mm in OD to <1.0 mm in OD) endoscope that (2) includes both imaging and illumination, (3) with a highly flexible shaft (multiple 360 degree tight turns with <5 mm bend radius without loss of performance), (4) short distal stiff tip (preferably <3 mm in length), (5) with pixel resolution from 3,000 pixels to more than 460,000 pixels, which is higher than VGA (depending on the pixel size used for the sensor and overall OD of the device construct disclosed in this application), (6) utilizes low cost software and hardware implementations for image reconstruction (<$500 even in small quantities) (7) has access ports for either gas or liquid flow to the distal end of the videoscope, and finally more importantly (8) has an extremely low-cost patient-contact portion (<$50 to <$20 for the complete imaging construct in high volumes: sensor, optics, cabling, illumination conduits, proximal connector, and assembly; a truly disposable, miniature, and highly flexible endoscope). Such a complete imaging tool not only can enable new procedures, but has the potential to improve and enhance existing medical devices and procedures by the addition of such construct in them.

It is finally the principal of this invention to describe how existing medical procedures, that currently do not include direct visualization, can greatly benefit by the addition of a miniature, highly-flexible, and disposable videoscope that will provide direct visualization to the physician.

### SUMMARY OF THE INVENTION

Although videoscopes for industrial or medical applications have been around for many years now, it is only recently that digital sensors have started to emerge with small enough overall footprints and high enough resolutions (due to smaller pixel sizes) that videoscope designs can be compatible with current mechanically demanding, miniature, and extremely low-cost requirements of minimally invasive procedures and medical devices.

In this patent application we define a videoscope as a construct that contains a digital imaging sensor in its distal end as well as provides illumination through the same conduit (like any typical endoscope). We further extend the definition to one that also offers access ports for delivery of fluids or gasses to the distal end of the interaction region. Such attractive properties render them practical for adaptation by either existing disposable medical equipment/devices or for the development of truly innovative, disposable, and minimally invasive medical devices that can offer direct visualization along with the delivery of some therapy and/or diagnosis.

It is the object of this application to highlight existing medical applications and medical devices that can greatly benefit from the incorporation of the above mentioned low cost, high resolution, and highly flexible endoscopic constructs.

Enteral nutrition is the preferred route for the provision of nutrition support in patients with a functional gastrointestinal tract. Soft, small bore feeding tubes are easily placed at the bedside, and have become the preferred method for providing temporary enteral nutrition access for acutely ill patients. It is estimated that more than 1.2 million small bore feeding tubes are used each year in the United States alone (Koopmann M C, Kudsk K A, Szotkowski M J, et al. “A Team-Based Protocol and Electromagnetic Technology Eliminate Feeding Tube Placement Complications,” Ann Surg., 253, 297-302, (2011)). Evidence accumulated over more than 25 years documents that between 1-2 percent of small bore feeding tubes that are placed blindly at the bedside enter the airway undetected, and a proportion of these misplacements result in pulmonary injury that is not preventable even by a single confirmatory radiograph (Woodall B H, Winfield D F, Bisset G S 3rd., “Inadvertent tracheobronchial placement of feeding tubes,” Radiology, 165, 727-729, (1987)). All available evidence suggests that blind placement of small bore feeding tubes is an unnecessary risk and should be abolished or enhanced with some form of active imaging to enhance patient safety. The need for some form of guidance or imaging becomes even more important should the need for the distal end of the tube to pass the pyloric sphincter for passage into the duodenum is necessary.

Syncro Medical Innovations Inc. has designed a magnetically guided feeding tube for such purpose (U.S. Pat. No. 6,173,199). Although an innovative approach to blind guiding, the operator still has small or very little feedback as to the exact location of the distal end of the feeding tube. Nonetheless the magnetic manipulation of the distal tip of a stylette insert allows easier access through the pyloric sphincter. But the approach still suffers from the uncertainty of a blind feeding tube placement. In a different study (Black et. al. in Chest, VOl 137, pg 1028-1032, 2010) the authors describe a method of attaching an endoscope with a clip on the outside distal end of a feeding tube, and then pushing both tubes either through the nose or the mouth of the patient for access into the stomach and then past the pyloric sphincter. Although the addition of imaging through the attachment of the scope alleviates the uncertainty of the exact location of the distal end of the feeding tube (compared to blind placement) this method of adding imaging to the placement of a feeding tube by default results in a construct that is larger than the outside diameter of the underlying feeding tube. This implies great patient discomfort, especially for transnasal access.

Some or all of these objects are achievable with the various embodiments disclosed herein. Additional objects, features and advantages of the various aspects of the present invention will be better understood from the following description of its preferred embodiments, which description should be taken in conjunction with the accompanying drawings.

### DESCRIPTION OF THE DRAWINGS

FIG. 1: Plot of the HyperForm Occlusion Balloon catheter made by EV3.

FIG. 2: Plot of the HyperGlide Occlusion Balloon made by EV3.

FIG. 3A: Picture of W.L. Gore and Associates Tri-Lobe Balloon catheter used for placement of AAA grafts.

FIG. 3B: View of FIG. 3A construct from its distal end. An AAA graft is also shown around the 3 inflated balloons.

FIG. 4: Schematic of the miniature imaging scope with a digital chip in the top picture and an OCFB in the bottom picture.

FIG. 5: Schematic of the imaging scope inserted in the feeding tube. Top picture with the scope attached in the ID wall of the of the feeding tube. Bottom picture the scope is an independent insert that can slide in and out of the feeding tube.

FIG. 6: Placement of the feeding tube and imaging scope transnasally all the way past the pyloric sphincter for post pyloric feeding.

### DESCRIPTION OF PREFERRED EMBODIMENTS

A videoscope with all the novel attributes described so far in this patent application can transform many exiting medical tools and procedures whose clinical benefit (safety and efficacy) can greatly be enhanced by the addition or use of such a videoscope or imaging in general.

In what follows we offer medical procedure applications that can greatly benefit by the incorporation of the miniature, flexible, and low cost imaging modalities described to this point and methods of incorporating such imaging in medical devices in order to enhance such medical procedures by the addition of direct visualization of the procedure or treatment area; direct visualization and imaging that is currently not available for such procedures or it is too expensive and cumbersome. All methods of use and applications described below constitute preferred embodiments of this patent application.

Suction Tools with Direct Imaging:

Suction tools are used routinely in numerous medical procedures. They typically comprise of a hollow tube with aspiration, so that one can better visualize in deep cavities, small openings, under flaps, and lateral margins. For example Invuity Inc. offers a handheld suction tool that they adapted with illumination in order to further assist the physician visualize while using such tools.

It is a preferred embodiment of this application to adopt any suction (aspiration tool with illumination and direct imaging. The imaging device can be any of the videoscope constructs disclosed here, or a fiberscope or any other miniature imaging device that could be attached onto an aspiration catheter without interfering with its primary function. Offering visualization with an imaging device at the distal end of the aspiration catheter could further assist the physician in performing the suction in a safer fashion without having to shift tissue around too much and thus make the procedure simpler and safer.

It is another embodiment of this patent application to enhance further the handheld illuminator (Agilux™ Handheld Illuminator) marketed by Invuity Inc. with an imaging device at the distal portion of such aspiration device. The addition of direct imaging would further improve the functionality of such device. Since the Invuity suction device already incorporated illumination, a scope with no illumination could be more than adequate for this device.

Upper and Lower GI Tract Imaging—Tethered Pill:

The miniature imaging scopes described here can be invaluable tools to any upper or lower GI medical device lumen that currently does not include direct visualization but depends on adjunctive endoscopy to provide the necessary visualization. Such addition can transform all these GI products to a simple and inexpensive (disposable) visualization-enabled medical device for the upper or lower GI tract, eliminating the addition of endoscopy for their use (thus also reducing the overall cost of the procedure since it would eliminate the upper or lower GI endoscopy charge/fee from the cost of the procedure).

In another embodiment of the device in this field, the videoscope can be envisioned as a tethered camera pill if it is connected with a long enough cable to cover the whole or a big portion of the whole GI tract. In this embodiment, unlike the pill camera products currently available (for example that offered by Given Imaging Ltd. (givenimaging.com/en-us/Patients/Pages/pagePatient.aspx for capsule endoscopy) that one swallows while the camera takes pictures of the whole GI tract, the physician can stop and monitor any region of the GI tract that seems suspicious with as much rigor and detail as necessary. Also unlike the pill camera access of the lower GI tract can also be achieved via a typical colonoscopy access as well.

Endovascular Microcatheters and Guidewire for Angioscopy Procedures:

Angioscopy for Placement or Assessment of Placement of Stents, Vascular Grafts or Coils:

With the proliferation of stenting, vascular graft, and coiling in endovascular procedures, the need to be able to directly visualize their placement during or at the end of the procedure or by re-entering the vasculature at a later time to assess the clinical situation at the pre-treated location is of importance to vascular interventionists. The imaging scopes described here can easily be incorporated into such construct. Just like the nasoenteral feeding tube, and depending on the endovascular application and size availability, the scope construct can be used as a compact videoscope (illumination and imaging bundled together in one construct) or as a composite (where illumination is separate from the imaging sensor and all are distributed around the circumference of the catheter).

Methods of Displacing Blood for Direct Endovascular Imaging:

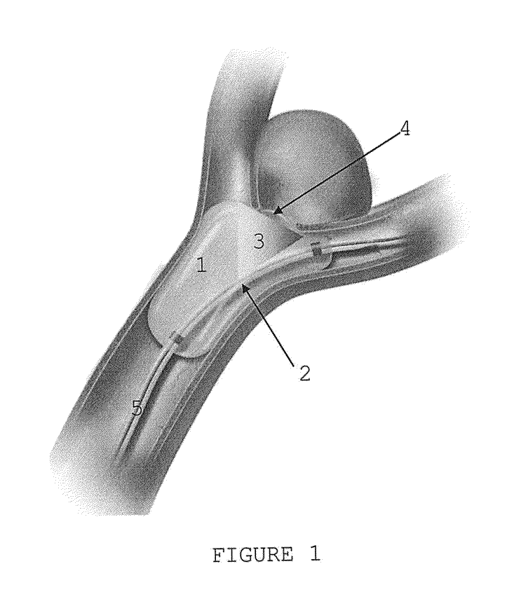

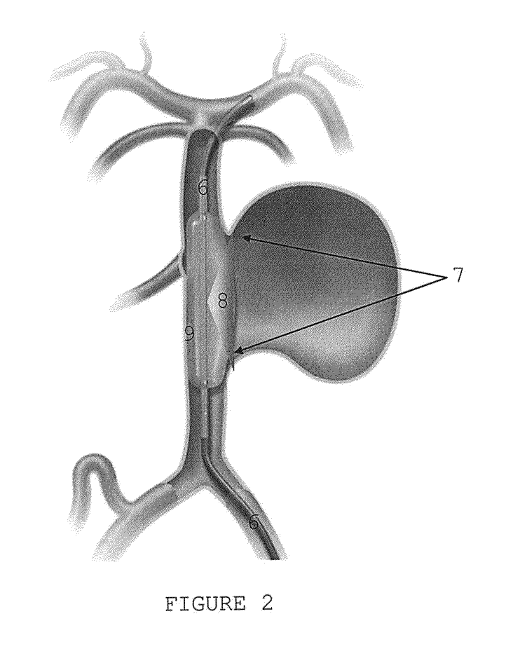

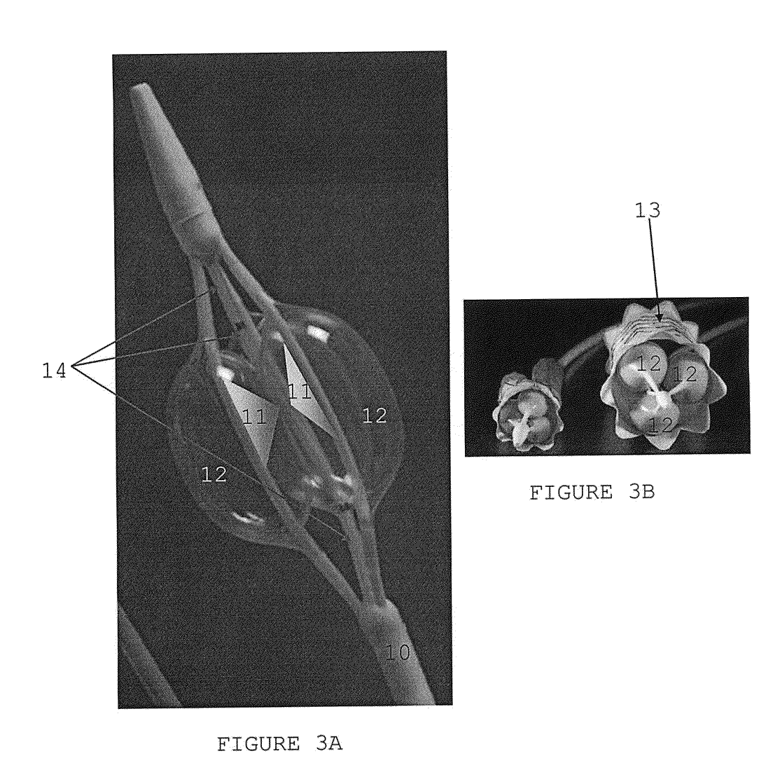

For direct visualization in the vasculature, some mechanism must be employed to displace the blood. This can be achieved by different methods. The ones listed below constitute different preferred embodiments of this application: (a) By a balloon at the distal end of the videoscope (where the imaging sensor resides) that is inflated by either gas or clear liquid. The balloon is inflated sufficiently to come in contact with the endothelial walls (or the walls of some other structure inside the endothelial wall, for example an already deployed stent or graft) of the vascular anatomy under consideration and in doing so, completely displace the blood from the field of view of the imaging device at the distal end of the videoscope/catheter. The imaging device can be inside the balloon with a clear view (through the insufflating balloon medium) of the surface that the outer balloon surface has come in contact with. Clearly in this method (and the other to follow) where a balloon is used to displace blood or arrest its flow, the balloon must be made of material that is transparent to the radiation used to perform the imaging. (b) By a proximal balloon on the videoscope catheter shaft (proximal to its distal tip or portion of the catheter that “houses” the imaging sensor) that is inflated briefly to arrest blood flow. A quick infusion of saline for example through the access ports of the disclosed videoscope can further clear any residual blood or other obstruction from the view of the digital sensor at the distal end of the videoscope. The compliant occlusion balloons (such as HyperForm or HyperGlide occlusion balloons) offered by EV3 (FIG. 1 and FIG. 2) or any other existing approved occlusion balloon catheters can be adopted or their overall designs can be adopted and enhanced by the addition of an imaging device with illumination distal to the proximal inflating balloon. (c) Without using a balloon, but by generating a column of clear liquid with a quick injection, for example of saline, through the access ports of the disclosed videoscope. The current fast frame rate miniature digital sensors can allow a brief real-time view of the area distal to its tip as the clear column of liquid passes through the field of view of the digital sensor. The advantage of this is that no timing or triggering mechanisms would be necessary, like some of the old-fashioned cumbersome angioscopy tools of the mid 90s (see for example R. A White and T. J. Fogarty editors “Peripheral Endovascular Interventions,” 2^{nd} edition, chapter 13, (1999)). By simply being able to collect images at fast frame rates (for example 45 frames per second) will ensure that the passage of the clear liquid column will be captured by the system; and thus images of the endovascular area of interest that was previously blocked by blood can be viewed and recorded. Note that for this disclosure a bolus injection of a clear liquid may also be performed by another catheter. For example a guiding catheter that the videoscope is going through, whose distal tip is proximal to the tip of the videoscope. Such clear liquid can be any liquid that is medically approved and optically clear, such as saline or contrast medium. (d) A special partial blocking balloon catheter similar to the Tri-Lobe balloon catheter design by W.L. Gore & Associates. The interesting geometry of the Tri-Lobe balloon in FIG. 3A is the fact that it is not completely occlusive when the balloons are inflated. The multitude of balloons come in contact with the vessel wall or graft wall, while the catheter simultaneously allows for blood to flow through the balloon structure (from the middle of the structure) and of course has a middle guidewire lumen for manipulation of the whole catheter.

We would like to point out that all four modalities for displacing blood are part of a disclosed embodiment of a catheter enhanced with imaging and illumination for endovascular direct visualization, and can be thought of as either a “host” catheter into which a videoscope construct previously disclosed is embedded in it, or as the new enhanced imaging catheter as a whole (or am enhanced videoscope).

Visualization of Aneurism Anatomy (Especially Wide Neck) and Deployment of Coils or Stents:

Addressing an aneurism in the vasculature with a minimally invasive catheter procedure consists of the deployment of an occlusive material that will occupy the void of the aneurism (such as a coil or epoxy) or a graft/stent looking structure that can block its “neck” into the vessel. All these procedures are currently done only under fluoroscopic visualization. In the spirit of this patent application, the tools used to deploy such vascular tools to treat aneurisms can be enhanced and modified with the addition of imaging. All these tools typically utilize an inflatable balloon, 1, that pushes up against the vessel wall when fully inflated. This action will naturally displace the blood from that area. If such catheter is equipped with an imaging device in its portion surrounded by a balloon, 2, the direct visualization of the treatment area can be made for the first time. This is a variation to the second method disclosed earlier for displacing blood for direct imaging utilizing a proximal inflating balloon; where this time the imaging sensor resides inside the proximal balloon (so it can view directly the portion of the surface the balloon comes in contact when it is inflated).

A sideviewing optic on the imaging device will be needed to view at some angle off the axis of the catheter (indicated by the triangular cone, 3, meant to indicate the field of view of the embedded optic) and into the neck of the aneurism, 4. The triangular cone, 3, is representative of a desirable viewing angle and direction for this geometry.

A multitude of cameras can be used inside the balloon situated so that they are looking at different spatial locations. For example the multitude of imaging sensors can be radially positioned around the catheter so that a panoramic 360 degree view of the vessel wall can be “stitched” or simply displayed as separate images. Such direct visual information along with fluoroscopy can provide unprecedented understanding of vascular disease and how it is currently treated.

A preferred embodiment of this device would be an enhanced version of the occlusion balloon catheters similar in form and function to the HyperForm and/or HyperGlide currently marketed by EV3, or any other existing balloon catheter utilized to deploy stents in the vasculature, or in general a balloon catheter that is similar in function with all the stent deployment or coil deployment balloon catheters currently marketed. But the disclosed proximal balloon catheter will contain one or more imaging device and illumination in them at the portion of the catheter surrounded by the inflated balloon.

The HyperForm Occlusion Balloon System is a highly conformable balloon that provides a soft balloon for interventional procedures. The balloon seals asymmetrical vasculature by forming nodes into surrounding branches. Such balloon catheter is ideal for bifurcations. In a preferred embodiment of this application, an imaging device can be embedded in the catheter portion of the HyperFlow-looking catheter that is surrounded by the occlusion balloon that is aiming in a direction such as that pointed out in FIG. 1. The balloon catheter, 5, is acting as a host to the imaging system. Any of the above disclosed videoscope designs, or a fiber scope, or any other miniature imaging device small enough to be hosted by such catheter can be the imaging device. Illumination must also be provided either in the form of illumination fibers or LEDs that either resides in the same shaft as the imaging system or are distributed independently along the host catheter. When the balloon is inflated and pushed up against the bifurcation walls, blood is displaced, and direct visualization of the relevant anatomy can be performed. Direct imaging of the aneurism neck, 4, or coil deployment can be easily made.

The HyperGlide Balloon provides accessibility to the distal vasculature and excellent trackability even through multiple turns. In a preferred embodiment of this application, an imaging device can be embedded in the catheter portion of the HyperGlide-looking catheter that is surrounded by the occlusion balloon that is aiming in a direction such as that pointed out in FIG. 2. The balloon catheter, 6, is acting as a host to the imaging system. A sideviewing optic on the imaging device will be needed to view almost at 90 degrees off of the axis of the catheter and into the neck of the aneurism, 7. The triangular cone, 8, is representative of a desirable viewing angle and direction for this geometry. Any of the above disclosed videoscope designs, or a fiber scope, or any other miniature imaging device small enough to be hosted by such catheter can be the imaging device. Illumination must also be provided either in the form of illumination fibers or LEDs that either resides in the same shaft as the imaging system or are distributed independently along the host catheter. When the balloon, 9, is inflated and pushed up against the bifurcation walls, blood is displaced, and direct visualization of the relevant anatomy can be performed. Direct imaging of the aneurism neck, 7, or coil deployment can be easily made.

We need to emphasize that the scope of the above mentioned embodiment of the device is not tied to the two aforementioned EV3 occlusion balloon models but to their overall design and function. They were mentioned earlier to aid the discussion. The scope of the disclosure encompasses all occlusion balloon catheters, or preferably their general design and function that uses an inflating balloon proximal to its distal tip to deploy a stent or coil.

Endovascular Imaging Through a Guidewire:

In this section we would like to capitalize on the concept of “miniature” and disclose a vascular imaging tool that comprises only of a guidewire without the need of using a balloon catheter. A small enough imaging sensor can be embedded directly into a guidewire. Such sensor can be a miniature version of the videoscope disclosed here or a miniature fiberscope or any other miniature imaging device that can fit inside the construct of a guide wire (which can typically be a metallic hollow tube for the most part). The imaging sensor can be positioned somewhere in the distal portion of the guidewire. Sideviewing optics may also be required on the imaging system of the sensor to facilitate viewing in a direction other than the main axis of the guidewire. Imaging in the vasculature can be performed with any of the previously four disclosed methods of displacing blood. Infusion of a column of clear liquid or inflation of a proximal balloon can be performed from a guide catheter that the guidewire is running through and is proximal to the interaction region viewed by the imaging sensor in the distal portion of the guidewire. In another embodiment of this application the guidewire can be additionally equipped with an inflation balloon. The imaging sensor can then reside in the portion of the guidewire that is surrounded by the inflating guidewire. Not requiring a balloon catheter, but rather inflating a balloon from with the guidewire, will tremendously simplify any endovascular procedure that requires balloon inflation. Direct visualization of the area that the balloon comes in contact with when inflated (by displacing the blood out of the view of the imaging sensor) adds yet another important clinical dimension to the utilization of such versatile quidewire.

Graft Placement Assessment:

The Tri-Lobe balloon by W.L. Gore, 10, is used for such task (adjustment and final placement of a graft) in Abdominal and Thoracic Aortic Aneurism procedures such as that of FIG. 3A. The two triangular cones, 11, indicate proposed placement of imaging devices within 2 of the 3 balloons, 12, and the direction of viewing of the imaging system. The third balloon is also fitted with a camera viewing out of the plane of the paper (cone not shown here). More than one imaging device per balloon may also be incorporated in this design. The guide wire lumen, 14, does not go through any of the balloons, 12, like it is usually the case for balloon catheters.

The benefit of this balloon catheter is that the multitude of balloons, 12, can come in contact with the graft, 13, and vessel wall (actually to modify and finalize its placement before complete deployment) while blood can continuously flow through the middle of the structure. By inflating the multitude of balloons in the Tri-Lobe catheter and making them come in contact with the vessel wall, one can manipulate the graft placement. At the moment, such final assessment is performed only fluoroscopically. The need for better imaging than the 2D images offered by an X-Ray Fluoroscope would greatly enhance the final decision about the placement of the graft.

It is a preferred embodiment of this patent application to disclose a modification to a multi-balloon catheter such as the Tri-Lobe Balloon catheter by W.L. Gore, where each balloon is retrofitted with one (or more) of the above mentioned videoscope structures, with possibly an added side-viewing optical element so that while the videoscope runs along the length of the catheter, it can view at some pre-determined angle off of the center axis directly the portion of the inside diameter of the vessel that the balloon comes in contact with. When the blood gets completely displaced, the balloon wall that comes in contact with the ID of the graft or vessel wall will come to view. The physician can make a direct visualization of the FOV area of the camera. Each balloon, 12, of the multi-balloon catheter can have one or more cameras, so the complete view of the area of interest can be displayed (stitched) on a screen or computer monitor for the physician who can make an assessment for the final placement of the graft. These direct visual images from the multitude of cameras inside the balloons along with the current visual aids from the fluoroscope can provide a far better perspective of the proper placement of the graft, and any anchors or other features of the graft. The camera and illumination fibers can be in separate structures and not necessarily in the same videoscope construct for as long as a camera and illumination fiber(s) are available for each of the 3 balloons of the Tri-Lobe balloon catheter design. In another embodiment of the device, where cameras and illumination exist in only one of the balloons, the physician must rotate the catheter in order to view all the areas of interest. LEDs may also be used to offer illumination at the distal end of each balloon. In another embodiment, each balloon can also be retrofitted with a regular fiberscope (instead of a digital camera), or the OCFB and the illumination fibers can be in different constructs (instead of one). In another embodiment of this design, more than 3 balloons can be available (especially if the FOV of the optics needs to be reduced) so that the camera in each balloon needs to be “responsible” to image a smaller portion of the inside circumference of the graft. In this embodiment of the imaging-enhanced Tri-Lobe Balloon Catheter, each balloon piece will have one or more than one imaging device and illumination. Imaging devices can be either the videoscopes disclosed earlier, or regular fibersocpes, or any other miniature videoscope or fiberscope technology that can form an image through the balloon. Illumination can be provided by a single or more than one fiber, or by LEDs that can effectively illuminate the inflated chamber of each balloon.

In another embodiment, we keep all the aspects disclosed earlier for the enhanced Tri-Lobe Balloon with imaging devices and illumination but we modify the structure of the balloons to be annular or donut shaped instead of axial. In this embodiment of this multi-balloon catheter, the balloons can be such that inflate in an annular shape instead of the axial shape that the Tri-Lobe Balloon by W.L. Gore currently has. More than 3 balloons can be part of this “donut-shaped” structure that radially comes in contact with the ID of the graft. As they get inflated, they displace blood, come in contact with the ID of the graft, while allowing a middle guidewire lumen and blood to flow through the middle of it. Each balloon piece will have one or more than one imaging device and illumination. Imaging devices can be either the videoscopes disclosed earlier, or regular fibersocpes, or any other miniature videoscope or fiberscope technology that can form an image through the balloon. Illumination can be provided by a single or more than one fiber, or by LEDs that can effectively illuminate the inflated chamber of each balloon.

In another embodiment of this application, the enhanced multi-balloon structures with imaging (that allows blood flow while deployed) can also be used to image stent or coil deployment/placement in the vasculature.

Finally we want to point out that this specific disclosure is not just tied to the Tri-Lobe Balloon architecture described earlier. Although the unique Tri-Lobe Balloon, 10, (W. L. Gore & Associates, Inc.) provides a dilatation force on three axes (separated by 120 degrees) in the aorta without complete blockage of blood flow, and reduces the windsock effect of the occluding balloon to the stent graft, other existing graft balloon architectures can be adopted by this disclosure as well. The concept of direct visualization through one or more imaging devices residing within an angioplasty balloon catheter is broad enough (and the essence of this disclosure) to be adopted by any angioplasty balloon design for assessing the placement of a graft in a aortic or thoracic AAA procedure, aid fluoroscopic visualization, and hopefully also reduce x-ray time (since some of the visualization can be performed directly with the imaging sensors instead with fluoroscopy). The Tri-Lobe design and architecture was used to aid the discussion and to disclose an embodiment. At the same time, for example, the Reliant Stent Graft Balloon designed to be used with the AneuRx AAAdvantage Stent Graft System (made by Medtronic, Inc.), is an excellent molding balloon for endografts as well. Same goes for Medtronic's Talent Xcelerant Hydro Delivery System, and Cook's Inc. Zenith system. Same goes for the CODA Balloon (Cook, Inc.), which is also very useful in endograft molding and aortic occlusion, and all other commercially available designs.

It is a preferred embodiment of this application to encompass all other Stent Graft Balloon designs and architectures as well as any other angioplasty balloon and enhance them with a miniature imaging device (or a multitude of imaging devices) and illumination for direct visualization of the area the surface of the expanding balloon comes in contact with when it is expanded, especially for assessing the placement of a AAA graft.

Direct Image Assisted Valvuloplasty:

Another vascular example that can be greatly enhanced by the addition of the disclosed videoscope or elements of it includes a valvuloplasty. Valvuloplasty is performed, in certain circumstances, to open a stenotic (stiff) heart valve. In valvuloplasty, a balloon catheter is advanced from a blood vessel in the groin through the aorta into the heart. Once the catheter is placed in the valve to be opened, a large balloon at the tip of the catheter is inflated until the leaflets (flaps) of the valve are opened. In the spirit of this section, the above mentioned balloon catheter can have the disclosed videoscope incorporated in it, or any other imaging device and corresponding illumination (such as a fiberscope or other miniature imaging devices that could fit in the catheter construct), so that when the balloon is inflated the blood is displaced and one can capture real video or numerous snapshots of the leaflets as they are pressed against the heart wall by the balloon wall. Once the valve has been opened, the balloon is deflated and the catheter is removed. Such visualization can offer unprecedented clinical data and value to the interventional cardiologists for the current patient condition and any possible future heart valve replacement surgery. For example the size and any possible deformation of the leaflets, or the amount of any sclerotic buildup on the leaflet could be easily assessed with the disclosed modality.

Enhancing and Complementing all Chronic Total Occlusion (CTO) Products and Atherectomy Devices for Peripheral Arterial Disease (PAD) by the Addition of Direct Imaging and Visualization Of the Treatment Region into Existing CTO and Atherectomy Products:

A CTO is defined as an artery that has been completely occluded for greater than 30 days. Chronically occluded coronary arteries account for approximately 20-30% of the documented coronary disease encountered in coronary catheterization labs today. Currently there are three methods for treatment of CTO's: percutaneous intervention, coronary artery bypass surgery (CABG) and medical management. Less than 10% of CTO cases are managed by percutaneous intervention. Approximately 40% of the CTOs are managed by surgical means and 50% by medications alone.

Medical therapy (e.g., nitrates, calcium, and beta blockers) is partially efficacious, but rarely completely eliminates either the symptoms or the objective evidence of the ischemia. Coronary Bypass Surgery is effective so long as the distal target vessel is anatomically suitable for insertion of a bypass graft. The limitations of the bypass surgery are well known and include significant patient morbidity, risk of surgical mortality, and significant expense.

The third option is percutaneous intervention. This minimally invasive, less costly procedure accounts for approximately 10% of coronary intervention cases. Percutaneous intervention is accomplished by using conventional guidewire techniques to slowly ‘poke’ and ‘prod’ through the occlusion. This procedure is successful 30-90% of the time depending on the operator skill and case selection criteria. The time spent to recanalize a chronic total occlusion is estimated to be between 5 minutes and several hours with an average time of about 30 minutes.

It is clear that in the case of a CTO, and a minimally invasive catheterization attempt to treat it, the physician must perform multiple random advancements of the guidewire in an attempt to blindly gain access of an entry point or orifice in the proximal end of the total occlusion so that the guidewire can be advanced through the occlusion and a stent or graft can be deployed in order to move it out of the way and open the occluded artery. This can be both dangerous and time consuming; both equally undesirable.

In the case of a CTO, the second method described earlier for displacing blood to gain endovascular access is the most preferable. The reason being, that the inflation of a proximal balloon is inconsequential since the artery is already occluded.

In a preferred embodiment of this patent application, a guidewire catheter with a proximal balloon is used as the host catheter, which is enhanced with an imaging device on or near its distal end and pointing in that direction as well. Such imaging device can be embedded into the host guidewire catheter and can be any of the videoscope constructs described in this patent application, or a fiberscope, or any other miniature and flexible imaging scope that can image the distal end of the host catheter and is small enough to fit in such catheter. Illumination must also be provided by either illumination fibers or LEDs that reside in the above mentioned imaging construct or are distributed independent of the imaging device in the host catheter. Either way, care must be taken for the illumination to be pointing in the same distal direction that the imaging device is aiming. Once the host catheter gets to the proximal end of the CTO, the proximal balloon can be inflated to completely block the blood. Saline can then be infused in the distal region between the inflated balloon and the proximal end of the CTO either through the access holes of the videoscope disclosed earlier or through access holes provided by the host catheter. Once the residual blood from the distal end of the catheter is displaced with saline, then the proximal end of the CTO can come to direct visualization. The physician, for the first time, can have direct view of the proximal end of the CTO and can now manipulate the guide wire under direct visual feedback in search of an opening or an orifice so it can be further pushed through the CTO to cross it. Once the CTO is crossed, the deployment of a balloon or stent or graft can easily be achieved to treat it. The proximal balloon can then be deflated, and the catheter removed.

In another preferred embodiment of the device, an appropriately scaled version of the above disclosed geometry and modality for CTO treatment can be used for any vascular anatomy suffering from a total occlusion that cannot be crossed with a guide wire easily, and where the interventionist needs to blindly push and retract and re-push the guidewire in hope that access can be gained through some orifice in the proximal end of the total occlusion so that the guide wire can cross it in order for the occlusion to be further treated. In this embodiment, any of the available atherectomy devices for PAV disease can be enhanced/adopted with the aforementioned imaging modality of the CTO treatment to greatly reduce risk of perforation and time of treatment, as well as for the first time provide invaluable direct visualization of the disease.

Replacement of IVUS:

Another endovascular application of embedding miniature cameras in existing medical devices is be the replacement of Intra-Vascular Ultra Sound (IVUS) imaging. In many vascular imaging applications, the large size of the IVUS probes prohibits the simultaneous use of the same vessel by both the IVUS sensor as well as the medical catheter that needs to get to the specific vascular anatomy of interest and perform a specific task. Thus some vascular imaging designs (not by choice) resort to passing the IVUS probe through a vessel that runs parallel or adjacent to the one that needs to be treated, while the specific treatment catheter tool runs through the treatment vessel. This way, one can utilize the ability of ultrasound to image well through tissue and visualize the distal location of the adjacent vessel where the treatment needs to be administered, without having the bulky IVUS tip interfere with the treatment catheter. The disadvantage of this approach is that it adds complexity and complication as the IVUS tip is passed through an adjacent vessel that under normal circumstances would not have to be interfered with or addressed by anything (especially a bulky IVUS catheter tip). In a preferred embodiment of this application, one can utilize the small size, flexibility, high resolution, and low cost attributes of the disclosed videoscope, along with either of the four endovascular modalities for displacing blood for direct visualization described earlier, and completely eliminate both the use of a bulky IVUS tip as well as unnecessary access of a healthy vessel adjacent to the one that needs to be treated. The disposable and highly flexible videoscope of this application, equipped with any of the aforementioned four endovascular imaging modalities described earlier can easily offer direct visualization anywhere in the vasculature and aid for any vasculature procedure that currently relies on IVUS for anatomical imaging information.

Enhancing RF Treatment of Renal Denervation (RDN) with the Addition of Direct Visualization of the Treated Renal Artery:

One of the body's primary methods for controlling blood pressure involves the sympathetic nervous system. This system includes the major organs that are responsible for regulating blood pressure: the brain, the heart, the kidney and the blood vessels themselves. One key player in long term blood pressure regulation is the kidney. Renal nerves communicate information from the kidney to the brain, and vice versa. In people with hypertension, the renal nerves are hyperactive, which raises blood pressure and contributes to heart, kidney and blood vessel damage. Selectively quieting hyperactive renal nerves causes a reduction in the kidneys' production of hormones that raise blood pressure and may protect the heart, kidney and blood vessels from further damage.

Ardian Inc. (now part of Medtronic) has proposed a novel method of achieving such renal nerve simulation to address among other things hypertension (see U.S. Pat. Nos. 7,617,005, 7,647,115, 7,873,417 fillings by Ardian Inc. among several). RDN treatment is currently investigational in the United States. Clinical use is initially focusing on hypertension, but the treatment is believed to demonstrate that the therapy will play a role in treating heart failure, insulin resistance and chronic kidney disease, diseases also characterized by a hyperactive sympathetic drive.

It is a preferred embodiment of this patent application to utilize any of the above mentioned imaging devices, modalities, methods and techniques of endovascular imaging to enhance the Ardian RF ablation catheter tip with an imaging device so that direct visualization of the renal artery treatment can take place during, after, or intermittently with the current RF treatment of the renal endothelial walls with the Ardian tip electrode or any other energy source or devices used to duplicate or improve what is currently done with the Ardian modality.

The clinical benefit of such enhancement can be tremendous as for the first time the treatment can utilize more than the typical resistive or single point temperature feedback mechanisms to monitor the advancement and efficacy of every treatment site within the renal arteries. For the first time one can actually monitor the direct denaturization of the tissue due to heating of the endothelial surface that the RF tip electrode of the Ardian catheter comes in contact with in real time. Depending on which method is adopted to enhance the existing RF treatment catheter with visualization, a wide angle viewing imaging system will allow direct visualization and monitoring of the tissue area that a single or multiple contact electrode tips make with the endothelium of the renal arteries. Also more than one camera may be used. The current Ardian modality could be enhanced with the saline injection modality of visualizing in the vasculature (disclosed earlier) to generate a column of clear liquid so that no balloon is involved. This will keep the new modified/enhanced catheter small, and simple. The proximal balloon modality for temporarily arresting blood flow may also be adopted. Also the partial balloon blocking modalities disclosed earlier (for graft placement) may be adopted here (miniaturized version of the Tri-Lobe, 10, architecture). In a further enhancement of that Ardian modality, RF electrodes are deposited on strategic locations on the outside surface of the balloons of the Tri-Lobe design, while the middle of the catheter can still allow for blood flow and a guide wire lumen that does not go through the balloons (basically a miniaturized version of the Gore Tri-Lobe Balloon, or the annular multiballoon alternative disclosed earlier, with RF electrodes applied on the outside surface of the balloons). In such modality more than one site could be treated at the same time (and monitored at the same time) since a multitude of electrodes can be deposited on strategic locations on the outside of the balloons, while a multitude of imaging devices with illumination could be monitoring all the active sites at the same time, while blood flow is not impeded completely. Applying the RF energy simultaneously on multiple sites can dramatically reduce the current treatment time (from up to 30 minutes or more to just a couple of minutes).

Such direct visualization not only offers the direct observation of the effects of heating on the endothelial wall of the renal arteries, but can also alert the physician of pending vaso-spasm that in general can be caused when catheter manipulation of the vasculature is involved. Such spasms can be extremely uncomfortable to the patient, as well as prolong the overall treatment time.

Modulation of Camera Viewing to Address Possible Interference with Other Energy Sources:

In what follows here we disclose an intermittent modality for miniature digital cameras with high frame rates (more than 5 to 10 frames per second). In the case that interference from other energy sources becomes a problem and affects the image quality of the sensor, one can modulate the camera so that it is not ON continuously; in essence reducing its frame rate. If having a high frame rate for imaging is not that important for the specific application, such modulation of the imaging sensor may be inconsequential, while at the same time it can address the interfering issue with the other energy source. In other words, while the camera is OFF (or while the display is frozen), one can apply or turn ON the other (interfering) energy source. This other interfering energy source may be for example Radio Frequency (RF) waves traveling through electrodes or liquids for tissue ablation, or heating. Such electrodes may run coaxially down the shaft of a catheter that also includes the aforementioned videoscope signal and power wires. In a preferred functional embodiment of the disclosed videoscope (in the case that it is embedded in a catheter shaft with a transmission line of an interfering energy source such as RF for example), if it is the case that application of the RF energy interferes with the image that gets transmitted by the digital sensor, one can multiplex the two so that when the RF is OFF, the camera is looking live, and when the RF is ON, the camera is disabled (or the image is disabled from viewing). In such preferred embodiment the timing of the ON and OFF cycles will be a function of the specific heating application for example. The OFF time of the RF energy source should be designed so that it is of the same order or magnitude or faster than the cooling rate of the treated tissue. A fast frame rate miniature digital camera should be able to accommodate that and provide a good balance so that any adverse effects from lengthening the treatment time can be minimized or even completely eliminated, while no significant delay is added to the way that live video is displayed to the end user.

Endovascular Imaging Utilizing IR Wavelengths WITHOUT Displacing Blood:

Water as well as whole blood have a very low absorption coefficient in the range between 700 nm to approximately 1,000 nm. This property can be used to propose a new modality of direct imaging in blood with a videoscope without the need of displacing the blood from the file of view of camera at the distal end of the videoscope. Such a modality would greatly simplify the overall imaging construct as there would be no need for balloons or infusion of clear liquids to displace the blood. It is the purpose of this section to disclose an embodiment of a videoscope that can visualize in blood without the need of displacing it from the field of view of the camera:

The Quantum Efficiency (QE) of the photosensitive material of typical CMOS sensors can easily extend out to 900 nm. There is measurable response at wavelengths higher than 700 nm. Furthermore, a color digital sensor can be manufactured without an IR blocking filter, which can enable the visualization of IR wavelengths at least for the red pixels (as the red pixel's transmission filter curve continues on at high levels past 700 nm; Although the Green or Blue filter data (for the green or blue pixels of a digital sensor) is not available to the public for wavelengths greater than 700 nm, it appears that their transmission starts increasing past 700 nm. If the illumination is provided by a mid-IR source, then there is no need for other filters, and a digital sensor with no IR blocking filter can be transformed to a Mid-IR imaging sensor. But if the illumination has any visible content (along with mid-IR) then one should utilize a low-wavelength blocking filter as the one described below: In this case, by placing a short wavelength blocking filter in front of a digital sensor (a blocking filter for all visible light below 700 nm but a high transmission filter for all light above 700 nm to at least until 900 or 1000 nm) one can transpose the digital sensor into an infrared camera in the range of at least 700 nm to 900 nm. Since blood and water transmit well in this wavelength range, in these circumstances, one can use the digital sensor for IR imaging in such wavelength range and thus view objects in blood without having to displace the blood from the field of view of the camera.

Of course illumination would have to be provided in the aforementioned Mid-IR wavelength range. Such illumination can easily be provided by mid-IR laser diodes or mid-IR LEDs. High-power (multi watt output power in CW mode) is readily available from many different commercial laser diode modules. Mid-IR laser diodes emitting at wavelengths higher than 700 nm are now very inexpensive and can easily be utilized as IR light sources even with the earlier mentioned PMMA fibers (since they can emit such high powers of laser output, they can easily overcome the increased attenuation loss of PMMA at wavelengths greater than 700 nm). Another choice of material for Mid-IR illumination fibers can be the polymer fibers made out of CYTOP. CYTOP is an Asahi Corporation polymer that is used to make the core material for polymer fiber optics and has very low attenuation for wavelengths greater than 700 nm and can provide a very efficient transmission line for illumination at such wavelengths. Finally any material that possesses enough transmission in the MID-IR region of interest described here, and introduces losses that can be overcome by increasing the output intensity of the light source on its proximal end while offering enough light at the distal end for the imaging sensor to form an image and while not being adversely affected by the higher levels of output light intensity of the source (in order to overcome the transmission loss of such fiberoptic material), is disclosed as a preferred material for the illumination fiber construct.

In a preferred embodiment of this modality, the digital camera should not have an IR blocking filter, and preferably would not have any of the RGB color filters (although as it was discussed earlier one could image in the wavelength range above 700 nm even if the RGB color filters are present; but probably not as efficiently). Illumination would be provided in the range of 700 nm-1,000 nm by a high-power mid-IR laser diode or LED via either PMMA, or CYTOP core fibers, or high NA glass-core with polymer cladding for a high-NA value fiber, or a standard glass fiber, or any other material that can transmit enough light to the distal end for the CMOS sensor to form an image. Of course in the case that the illumination is provided strictly by a mid-IR source, the low wavelength blocking filter (for wavelengths lower than 700 nm) would not be necessary. But if the illumination source has visible wavelength content (wavelengths lower than 700 nm) one should add a short wavelength blocking filter in front of the camera to block such visible wavelengths and only transmit mid-IR wavelengths (700 nm and above). Depending on the geometry of the medical endovascular procedure, side viewing optics may also have to be added to the Mid-IR imaging videoscope so that the camera can image objects that are at some angle other than the normal to the surface of the sensor.

Clearly this method of directly imaging in blood without having to displace it should also be thought of as yet another alternative method of imaging for all the aforementioned endovascular procedures. It is a preferred embodiment of this patent application to encompass such modality of MID-IR imaging with regular CMOS cameras and further simplify all the other methods disclosed earlier where a balloon had to be deployed to displace the blood or a clear liquid injection had to be made to generate a column of clear liquid that the camera could see through.

General Endovascular Imaging—Reduction of Radiation Exposure:

The current patent application should not be limited to adding imaging to a specific endovascular procedure, but it encompasses any and all endovascular procedures that can greatly benefit (in efficacy or safety) from the addition of direct visualization in addition to any fluoroscopic guidance or visualization. Furthermore, the total amount of radiation that both doctors and patients are exposed to during minimally invasive endovascular procedures under fluoroscopy has been the concern of the interventional radiology community in general. Novel methods of assisting these vascular procedures in reducing such exposure would be greatly welcome. The addition of direct imaging to endovascular tools could possibly eliminate fluoroscopy, but at minimum, highly reduce the amount of time it is used during a procedure (since some of the imaging can be performed under the direct visualization methods disclosed earlier in this patent application). It is an embodiment of this patent application the use of any of the above mentioned vascular imaging techniques of direct optical visualization in blood to enhance any vascular procedure that can clinically benefit from the addition of such direct optical imaging and reduce or eliminate the amount of fluoroscopy applied to the patient, and amount or radiation exposure to the patient and physicians in the angioscopy suite.

Nasoenteral Feeding Tubes with Direct Imaging:

The ability of adding a miniature (no significant effects on the overall size of existing tubes), highly flexible (no adverse effects on the mechanical properties of existing feeding tubes), and extremely low cost (disposable constructs that can be disposed after single use along with the feeding tube) direct visualization solution such as the disclosed videoscope operating at the distal end of a nasoenteral feeding tube can offer a positive confirmation of the path of the tube during insertion and completely eliminate all the aforementioned complications of blind delivery.

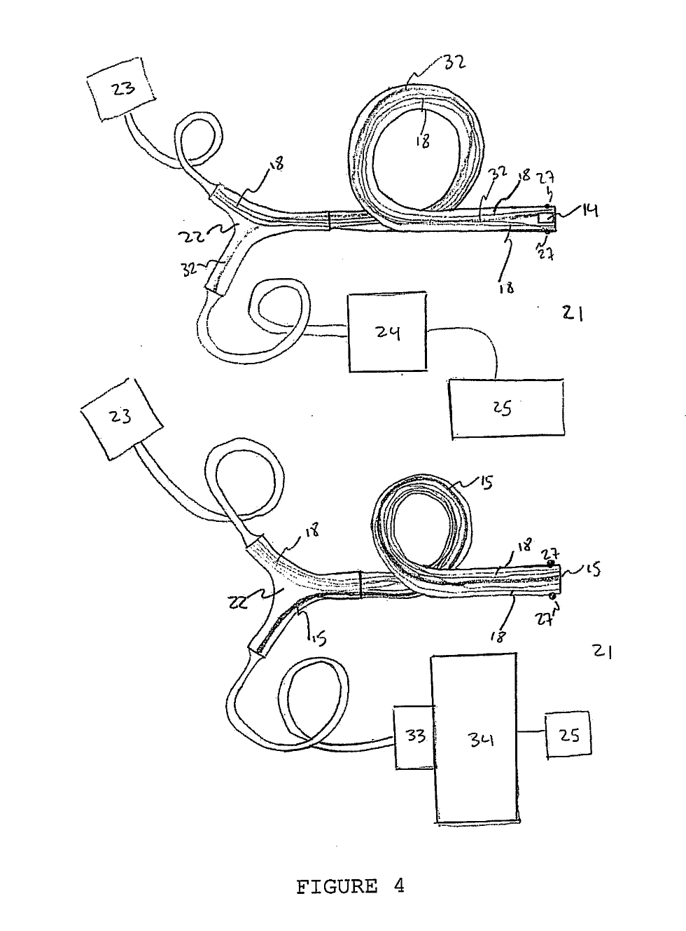

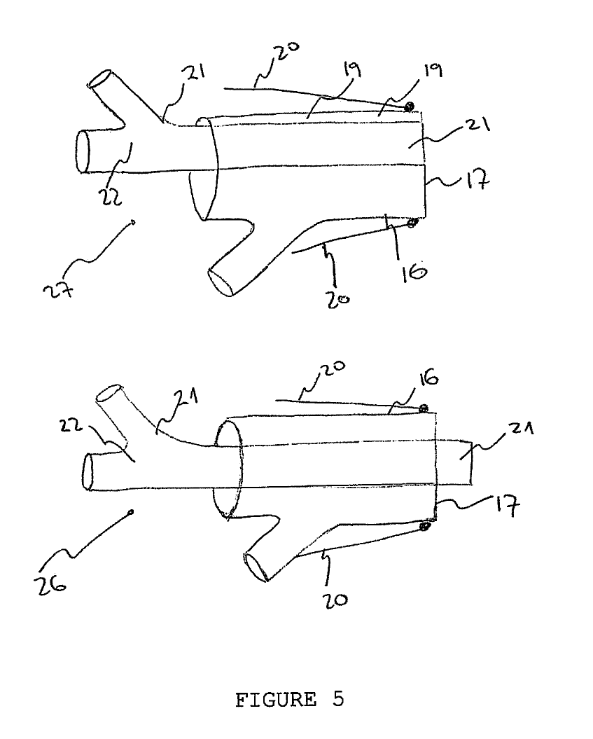

An embodiment of this patent application can be an approximately 1 mm OD (3 Fr-4 Fr) imaging scope 21 construct, shown schematically in FIGS. 4 and 5, that comprises either a video camera 14 or a coherent imaging bundle 15. Illumination can be provided by fibers 18 that run along the length of the scope. The scope can be attached on the ID (or OD) of an existing feeding tube 16, preferably at the ID of the feeding tube, as at 19. Its presence will not much affect the volume opening distal end 17 of a typical 12 FR feeding tubing and certainly it will not affect its mechanical properties or cost in any adverse way (for a truly disposable imaging system). The video camera cabling 32 and illumination fibers are split at the proximal end of the imaging scope in a Y-fork shape 22 so that illumination fiber can proceed to the light source 23, and the video cable to an image processing board 24 and eventually to a monitor 25.

Furthermore, in another embodiment, the illumination fibers can be separated from the imaging sensor. In other words the illumination fibers and the imaging sensor do not have to be together in one shaft. In another embodiment of this application the camera and its cabling as well as a multitude of illumination fibers can be positioned circumferentially on the distal round end of the feeding tube so that the overall volume occupied by the elements of the videoscope (illumination fibers and imaging sensor) can be almost evenly distributed around the circumference of the feeding tube instead of all being bunched together in one spot.

All the feeding tube constructs described so far can include articulation of the distal end of the feeding tube, 20. To those knowledgeable in the art such articulation can be achieved by the simple manipulation of the proximal ends of 3 or 4 wires that run along the length of the feeding tube, 20, and are attached only near its distal end. The wires can be internal or external.

All of the above mentioned uses of a videoscope with illumination fibers in a feeding tube can be duplicated with a fiberscope and illumination fibers. Thus in another embodiments of this application, the direct imaging addition to the feeding tube can be accomplished with an OCFB 15, as well (instead of a digital camera). In this case the image processing board 24, and monitor 25 can be replaced by an eye piece 33 attached directly on the proximal end of the OCFB, and a camera 34 that is attached onto the eyepiece to generate an image onto a monitor.

In another embodiment of the device, the imaging sensor and the illumination necessary for it can be a separate insert 26, like a stylette. Thus one can use a miniature videoscope 21, for example with an overall ID<2 mm that includes a digital sensor and illumination fibers (the imaging stylette) that can be inserted through an off-the-shelf feeding tube. In this example, the micro endoscope insert can be used for just the purpose of providing continuous imaging of the distal end of the feeding tube as the operator is inserting it into the body. In such design the articulation wires can also be attached onto the imaging scope insert 27. Once the feeding is placed correctly, 28, the imaging stylette can be removed and the feeding can commence.

Since feeding tubes are inherently larger medical shafts (compared for example to the sizes of some more delicate endovascular catheters), illumination can be provided by LED's instead of illumination fibers. Thus in other embodiments of the feeding tube imaging enhancement, the above mentioned videoscope or fiberscope constructs can be used with LED's instead of illumination fibers. In this case the illumination fibers 18 are eliminated, and the light source 23 is replaced by an LED driver.

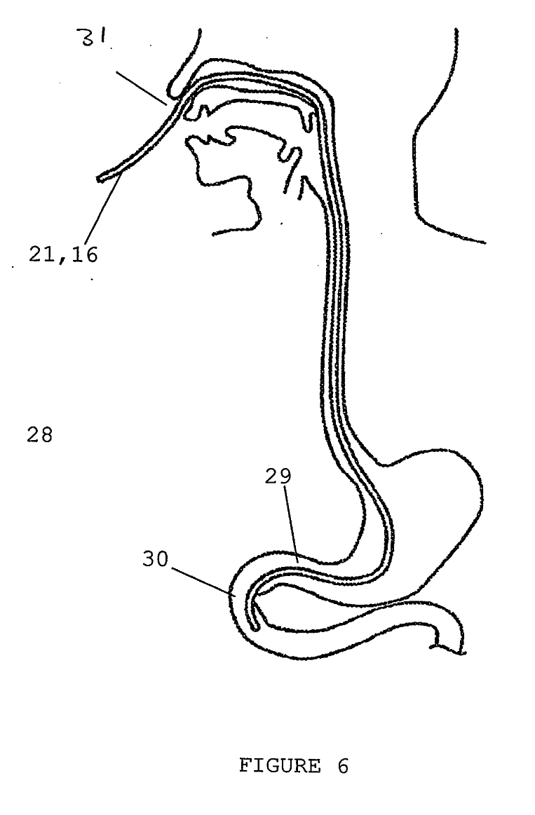

Finally, direct imaging of feeding tube placement can assist tremendously in the cases that require trans-pyloric enteral tube feeding as indicated in FIG. 6. Access to the stomach and beyond can be achieved transnasally, 31, or through the mouth. Assessing the location of the pyloric sphincter 30 at the distal end of the stomach 29, and guiding the feeding tube through it under continuous visualization is a trivial task with the constructs described herein.

General Use:

All of the above mentioned examples have one major thing in common. They all prescribe a general method of use for a miniature image scope, where a great amount of clinical value or safety value can be added in existing medical tools that currently do not incorporate direct visualization. It is the purpose of this patent application to include all existing medical tools that currently do not incorporate direct visualization but can greatly benefit (either clinically or by increasing their safety) by the addition of the disclosed videoscope construct or any other miniature imaging device that satisfies similar specifications as the disclosed videoscope in them, or by the addition of elements of the disclosed videoscope; where the camera and illumination fibers are not held together in the same shaft but are distributed along the axis of the medical device to which direct visualization is added.

The above described preferred embodiments are intended to illustrate the principles of the invention, but not to limit its scope. Other embodiments and variations to these preferred embodiments will be apparent to those skilled in the art and may be made without departing from the spirit and scope of the invention as defined in the following claims.

We claim:

1. A feeding tube with a videoscope, for visual guidance of the feeding tube into a patient, comprising:
a flexible feeding tube suitable for nasal or oral insertion for enteral feeding of a patient,
an endoscope within the feeding tube and passing through the length of the tube, with illumination means for lighting a path of the tube into the patient and image means for providing at the proximal end of the tube a continuous image of patient tissue at the distal end of the tube as the tube is advanced into the patient, and
the endoscope being removable from the feeding tube after placement of the feeding tube,
whereby the endoscope assists an operator in properly placing the feeding tube through the esophagus, as well as assisting in placement of the tube past the pyloric sphincter when post pyloric feeding is needed.

2. The feeding tube of claim 1, wherein the illumination means comprises at least one optical fiber carrying light from the proximal end to the distal end of the tube.

3. The feeding tube of claim 1, wherein the illumination means comprises an LED at the distal end of the tube, with electrical wiring extending from the LED to the proximal end of the tube.

4. The feeding tube of claim 1, wherein the image means comprises at least one optical fiber conveying images from the distal to the proximal end of the tube.

5. The feeding tube of claim 4, wherein the endoscope comprises a videoscope, with a camera at the proximal end of the optical fiber producing a digital image displayed on a monitor.

6. The feeding tube of claim 1, wherein the video means comprises a digital camera at the distal end of the tube, with electrical wiring connecting the camera to the proximal end of the tube.

7. The feeding tube of claim 1, further including a plurality of steering wires connected to a distal end of the endoscope and extending to the proximal end of the feeding tube, providing an operator ability to steer the distal end of the feeding tube.

8. The feeding tube of claim 4, further including a plurality of steering wires connected to a distal end of the endoscope and extending to the proximal end of the feeding tube, providing an operator ability to steer the distal end of the feeding tube.

9. The feeding tube of claim 5, further including a plurality of steering wires connected to a distal end of the endoscope and extending to the proximal end of the feeding tube, providing an operator ability to steer the distal end of the feeding tube.

10. A method for proper placement of a feeding tube in a patient, comprising:
inserting through the length of a feeding tube, to a distal end of the feeding tube, a flexible endoscope such that the endoscope extends to the proximal end of the feeding tube, and the endoscope including illumination means for lighting a path into the patient,
with the endoscope within the feeding tube, inserting the feeding tube into the patient nasally or orally and, with the illumination means, lighting a path of the tube through the patient and viewing the path from the proximal end of the endoscope while advancing the tube, and properly advancing and steering the tube into the esophagus and, if post pyloric feeding is needed, past the pyloric sphincter.

11. The method of claim 10, wherein the endoscope comprises a videoscope, and including viewing the path of the tube on a video monitor forming a part of the videoscope.

12. The method of claim 10, wherein the endoscope includes a plurality of wires secured to a distal end of the endoscope and extending to the proximal end of the feeding tube, and including steering the distal end of the tube as the tube is advanced, by applying tension to selected ones of the wires, based on visual information provided by the endoscope.

13. The method of claim 10, further including the step of pulling back and removing the endoscope from the feeding tube after proper placement of the tube. proximal end to the distal end of the stylette or white light LEDs at the distal end of the stylette for illumination. Such stylette can be inserted though a standard enteral feeding tube so that it can provide continuous imaging of the location of the distal end of the feeding tube as it is being inserted in the human body by the operator (through the nose or mouth of the patient). Such continuous visualization can assist the operator in providing proper placement of the feeding tube through the esophagus as well as assist in placement of the feeding tube past the pyloric sphincter for post pyloric feeding. Thus the imaging scope stylette can be used as a guide to assist in the proper placement of the feeding tube.
